(19) United States
(12) Patent Application Publication
(10) Pub. No.: US 2004/0091862 A1
(43) Pub. Date: May 13, 2004

(54) Method and device for detecting temperature-dependent parameters, such as the association/dissociation parameters and/or the equilibrium constant of complexes comprising at least two components

(76) Inventors: Albrecht Brandenburg, March (DE); Hans-Peter Lehr, Freiburg (DE); Holger Klapproth, Freiburg (DE); Meike Reimann, Freiburg (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/181,745

(22) PCT Filed: Jan. 22, 2001
(86) PCT No.: PCT/EP01/00664

(30) Foreign Application Priority Data
Jan. 21, 2000 (DE) 100 02 566.8

(51) Int. Cl. C12Q 1/68; G01N 33/53; C12M 1/34; G01J 3/00

(52) U.S. Cl. 435/6; 435/7.1; 435/287.2; 356/300

(57) ABSTRACT

The present invention relates to a method and a device for determining temperature-dependent parameters, such as the association/dissociation parameters and/or the equilibrium constant of complexes that comprise at least two components, wherein the first components, which are in a liquid phase, are contacted with measuring points located preferably on a planar optical waveguide of a reaction carrier and formed by second components linked to the solid reaction carrier and specifically binding to said first components, with the aid of a preferably heatable means for contacting the liquid phase and the reaction carrier under formation of complexes. Fluorescent dyes bound to the first components and/or the second components are excited in the surface area of the planar optical waveguide, preferably by the evanescent field of excitation light coupled into the planar optical waveguide, for emitting fluorescent light. Detection of the emitted fluorescent light takes place in the surroundings of the optical waveguide. The formation or the dissociation of the complexes comprising first components and second components is observed as a function of temperature.

The present invention relates to a method and a device for determining temperature-dependent parameters, such as the association/dissociation parameters and/or the equilibrium constant of complexes that comprise at least two components, wherein first components, which are in a liquid phase; are contacted with measuring points located on an optically excitable reaction carrier and formed by second components linked to the solid reaction carrier and specifically binding to said first components, and wherein a fluorescent light is produced by radiating in excitation light, especially laser light, which is evaluated via a detection means.

In biological and chemical systems the formation (association) and the decomposition (dissociation) of complexes is relevant. For example, the blood-sugar level is controlled by the binding of insulin to its cellular receptor, i.e. by the formation of an insulin/receptor complex. The complex formed leads to a reduction of the blood-sugar level. Other examples of complexes In biological systems are e.g. antigen/antibody and enzyme/substrate complexes.

For the biological and the chemical activity of the complex, both the kinetic parameters, i.e. the association as well as the dissociation constants, and the thermodynamic parameters, i.e. the equilibrium constant, are relevant. The temperature-dependence of the above-mentioned magnitudes is known.

The naturally occurring deoxyribonucleic acid (DNA) normally occurs as a double strand, i.e. as a complex of two complementary nucleic acid single strands. The rate of replication and transcription processes strongly depends on the distribution between complex and single strands.

The dissociation of the complex into two separate single strands is normally referred to as “melting” and the temperature at which approx. 50% of the complex have dissociated into the separate single strands is referred to as “melting temperature”. Generally, the tendency towards melting of the complex increases as the temperature increases.

For determining substances in samples, especially for determining specific DNA sequences In a sample, the use of biochips is known. These biochips form planar substance carriers on the surfac of which a plurality of measuring points, which are formed e.g. by nucleic acids (complementary DNA strands), are immobilized, said chip surface being contacted with a sample containing the DNA sequences as substances to be analyzed and the sample containing the nucleic acids to be analyzed. Since each single strand of a nucleic acid molecule binds to its complementary strand, this binding being referred to as hybridization, information on the DNA sequences contained in the sample will be obtained when the individual measuring points have been examined with respect to the binding of sample molecules. One of the advantages of biochip analytics is that up to a few thousand hybridization events can be carried out and detected in parallel on one biochip.

In accordance with the parallelism of the hybridization events, an analyzer for evaluating the biochip is necessary, which achieves both a high local resolution and also a high sensitivity of detection. Since the outlay required for pretreating the samples should be kept as small as possible, it is additionally necessary that even a small number of hybridized molecules is still reliably detected at the individual measuring points.

Biochip readers which are nowadays commercially available operate according to the scanning principle. The light used for exciting fluorescence serially scans the surface. The biochip is either moved rapidly relative to a fixed light beam or a galvano-scanner is used, at least for one direction of movement, by means of which the light beam is deflected. The light emitted by the fluorochromes is then detected by a sensitive photodetector (e.g. a photomultiplier). The devices are implemented as laboratory measurement systems for use In the field of molecular-biological research. The detection limit is in the range of a few molecules per μm² up to approx. 100 per μm². The scanning times range from approx. 2 to 4 minutes. The costs for such devices range from approx. 50,000 US-$ for a reasonably-priced device to approx. 350,000 US-$ for devices of higher quality.

By way of example, the two devices which are presumably most wide-spread today are here discussed in detail. These devices are the GeneArray Scanner produced by Hewlett Packard and sold by the American firm Affymetrix, and the ScanArray 3000 of GSI Lumonics. The GeneArray Scanner optically scans the chip surface by fast deflection of the light beam in one spatial direction. In the other direction, the chip is moved step by step. The dimensions of the device are 66cm×78 cm×42 cm. The light source is an argon ion laser (wavelength 488 nm). Detection is carried out by means of a photomultiplier at 550 to 600 nm. The ScanArray 3000 is provided with a fixed optical system. The scanning process is realized by a fast movement of the chip in one spatial direction and a step-by-step displacement in the other direction. Up to three different excitation wavelengths are offered for exciting various fluorochromes. Detection is carried out by means of a photomultiplier also in this case. All the measuring devices evaluate the biochip when the hybridization has been finished.

However, one feature which all these devices have in common is that they are incapable of detecting the temperature dependence of relevant kinetic and thermodynamic parameters, such as the association and dissociation constants and the equilibrium constant.

in addition, these devices entail problems in the case of a parallel measurement of the melting point of a nucleic acid hybrid having one strand immobilized on a solid phase. The melting point of partially complementary nucleic acids can only be calculated very inaccurately mathematically, especially when reaction partners bound to a solid phase restrict the degrees of freedom of the reaction.

A further problem inherent in these devices is the determination of the hybridization kinetics of complex samples for analyzing and for determining the concentration of a plurality of nucleic acids in a substance to be analyzed. The detection of multiple nucleic acids by hybridization is limited by the melting point problem of the hybrids. If the actual melting points of the hybrids, which often deviate from the calculated melting points, do not the within a close temperature range, this may disturb a measurement qualitatively, i.e. the measurement may be wrong-negative or wrong-positive, and also in the quantitative region.

It is therefore the object of the present invention to improve a method and a device of the type mentioned at the start in such a way that a precise determination of temperature-dependent parameters, such as the association/dissociation parameters and/or the equilibrium constant, is made possible in a simple way and without high demands on the pretreatment of samples.

According to the present invention, this object is achieved by a method of determining temperature-dependent parameters, such as the association/dissociation parameters and/or the equilibrium constant of complexes that comprise at least two components, wherein the first components, which are in a liquid phase, are contacted with measuring points located on an optically excitable reaction carrier and formed by second components linked to the solid reaction carrier and specifically binding to said first components, under formation of complexes, wherein the excitation of fluorescent dyes which are bound to the first components and/or the second components and which are located close to the surface is effected by transmitted excitation light so that fluorescent light will be emitted, and the detection of the emitted fluorescent light takes place in a variable temperature field, and wherein the formation or the dissociation of the complexes comprising first components and second components is observed as a function of temperature.

In accordance with a preferred embodiment of the method according to the present invention the first and/or second components are receptors and/or ligands.

In the present context, the expression “ligand” stands for a molecule which binds to a specific receptor. Ligands comprise, among other substances, agonists and antagonists for cellular membrane receptors, toxins, toxic biological substances, viral epitopes, hormones (e.g. opiates, steroids), hormone receptors, peptides, enzymes, enzyme substrates, cofactors, medicinal substances, lectins, sugar, oligonucleotides, nucleic acids, oligosaccharides, proteins and antibodies.

In the present context, the expression “receptors” stands for a molecule having an affinity for a ligand. Receptors may be naturally occurring or synthetically produced receptors. Receptors may be used as monomers or as heteromultimers in the form of aggregates together with other receptors. Exemplary receptors comprise agonists and antagonists for cellular membrane receptors, toxins, toxic biological substances, viral epitopes, hormones (e.g. opiates, steroids), hormone receptors, peptides, enzymes, enzyme substrates, cofactors, medicinal substances, lectins, sugar, oligonucleotides, nucleic acids, oligosaccharides, cells, cell fragments, tissue fragments, proteins and antibodies.

The ligands and/or receptors may be bound covalently or non-covalently to the reaction carrier. Binding can take place in the manner known to the person skilled in the art.

In accordance with another preferred embodiment, the first and/or second components are nucleic acid single strands. According to a specially preferred embodiment, the nucleic acid single strands are at least partially complementary to one another. The at least partially complementary nucleic acid single strands form, under suitable conditions, a nucleic acid hybrid. The temperature-dependent binding as well as the dissociation (melting) of the nucleic acid hybrid can be determined by the method according to the present invention.

On the basis of the exact knowledge of the melting points of nucleic acid complexes, it is possible to substantially Improve the mutation analysis on biochips. Hence, measurement data for a rational probe design for nucleic acid analytics can be developed. This will especially permit the use of probes for a parallel isothermic analysis.

In this connection it will be of advantage when the excitation light is coupled into the optical waveguide with the aid of optical means, such as a prism.

Furthermore, the excitation light may, by total reflection (ATR) or total internal reflection fluorescence (TIRF) of the light beams at an Interface between two media having different optical thicknesses, produce an electromagnetic field in the optically lighter medium, the optically denser medium being a solid phase and the optically lighter medium a liquid phase, for measuring the temporal progress of the reaction.

Such a method permits a very simple design of the reaction carrier, especially of the biochip, preferably by coating a transparent body with a planar waveguide layer having a high refractive index, and It permits a simple analysis device in the case of which e.g. a flow cell (or a cuvette or some other stationary sample body) containing the sample is brought into sealing surface contact with the reaction carrier, especially the biochip, whose surface is implemented, at least essentially, as a planar waveguide and guides the excitation light which is coupled Into said planar waveguide at one end thereof, the fluorescent light excited by the evanescent field of the excitation light being detected through the transparent carrier body on the side of the reaction carrier or biochip located opposite the planar waveguide and the flow cell by means of an optical imaging system, which preferably includes a filter, and being supplied to an associated spatially resolving detector, e.g. a photomultiplier or a CCD camera, for reading the reaction carrier, especially the biochip.

In so doing, all the measuring points on the upper surface of the planar waveguide of the reaction carrier or biochip are excited simultaneously by the evanescent field of the excitation light coupled into the planar optical waveguide for fluorescent light emission in combination with a reaction between the immobilized agents on the chip surface, such as DNA single strands, and the substances to be detected in the sample, such as DNA single strands.

Further preferred embodiments of the method according to the present invention are specified in the rest of the subclaims.

As far as the device is concerned, the above-mentioned object is additionally achieved in accordance with the present Invention by a device for determining temperature-dependent parameters, such as the association/dissociation parameters and/or the equilibrium constant of complexes that comprise at least two components, said device comprising a reaction carrier whose optically excitable surface is provided with second components specifically binding to the first components and forming measuring points on said reaction carrier, a device for contacting the first components, which are in the liquid phase, and the second components, which are linked to the reaction carrier and which specifically bind to said first components, a means for bringing the measuring points to a specified temperature range, a light source for coupling in excitation light so as to excite the emission of fluorescent light in dependence upon the binding of said first components to said second components of the reaction carrier, and a detector for detecting the emitted fluorescent light so as to determine the binding of said first components to said second components as a function of temperature.

In addition to the exact determination of the above-mentioned melting points, such a device can also be used for measuring the dissociation/association kinetics of nucleic acids via a temperature gradient, and, on the basis of the calculable equilibrium constants, it can now provide clear information on the reaction enthalpy and, consequently, permit a concentration determination of the substances to be analyzed. Even more complex hybridization curves, e.g. a plurality of hybridization partners at one probe—this is a nucleic acid bound to the solid phase—can be evaluated by a mathematical analysis of the hybridization curve so that parameters, which could not be established by means of a chip up to now, can now be detected with the aid of this analysis method.

In addition to the reading of biochemical reactions, the device according to the present invention is also suitable for detecting inorganic substances. One example for this kind of use is the gas sensor technology.

In the past, various gas sensors have been suggested, which use so-called sensitive coatings. These coatings may e.g. be polymer films or sol-gel layers which absorb certain gases. When this layer has incorporated therein substances reacting specifically with the analyte and/or indicators, a change of a layer property in the presence of certain gases can be detected. The layer properties in question may e.g. be a change of colour, a change of density or refractive index, or a change of the dielectric properties.

The device according to the present Invention can be used for gas detection in a similar way, when either the analyte fluoresces or when a fluorescence of the coating is suppressed by absorption of the analyte (“fluorescence quenching”). The optical arrangement according to the present invention permits in these cases the detection of a large number of analytes, when a plurality of different coatings is used, which are applied to the surface of the waveguide or of the prism in a patterned form. In addition, the temporal progress of the reaction is detected.

An important additional information is obtained from the temperature dependence of the gas absorption of the coating, since, at an elevated temperature, the gases to be detected will normally desorb. The knowledge of the temperature at which only a certain, determinable percentage of the gas concentration is still contained in the film increases the specificity of the sensor. This information can, however, also provide Information about the condition of the layer (e.g. ageing of the layer). The measurement data will become more reliable in this way. In particular, it is possible to detect the sensor data independently of absolute fluorescence intensities. In the case of a continuous or quasicontinuous measurement, the increase in temperature offers, last but not least, the possibility of driving out of tile layer also gases which do not desorb automatically when the ambient concentration decreases. The fluorescence measurement carried out simultaneously makes known whether desorption of the gas has taken place.

The gist of the invention is to be seen in the determination of temperature-dependent parameters, such as association/dissociation parameters and/or the equilibrium constant of complexes that comprise at least two components, wherein the first components, which are in a liquid phase, are contacted with measuring points located on an optically excitable reaction carrier and formed by second components linked to the solid reaction carrier and specifically binding to said first components, and wherein the formation or the dissociation of the complexes is observed as a function of temperature.

Further preferred embodiments of the device according to the present invention are specified in the rest of the subclaims.

In the following, the present invention will be explained in detail making reference to embodiments and the associated drawings, in which:

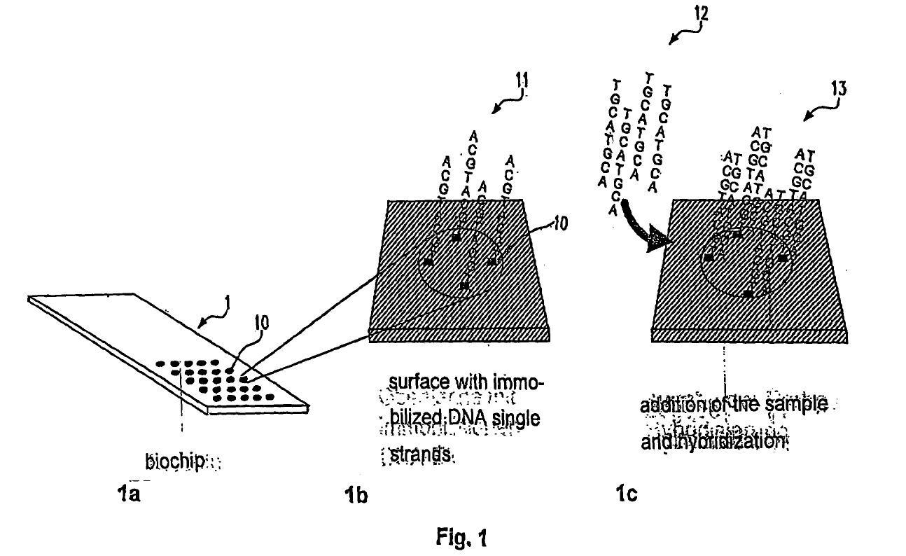

FIG. 1a shows a biochip in a schematic perspective representation;

FIG. 1b shows a detail of a biochip according to FIG. 1a for a measuring point of a surface with immobilized DNA single strands;

FIG. 1c shows a schematic representation of the addition of the sample with the DNA single strands to be analyzed to the measuring point according to FIG. 1a, and a representation of the complementary interaction between the immobilized DNA single strands according to FIG. 1b and the DNA single strands contained in the sample (hybridization);

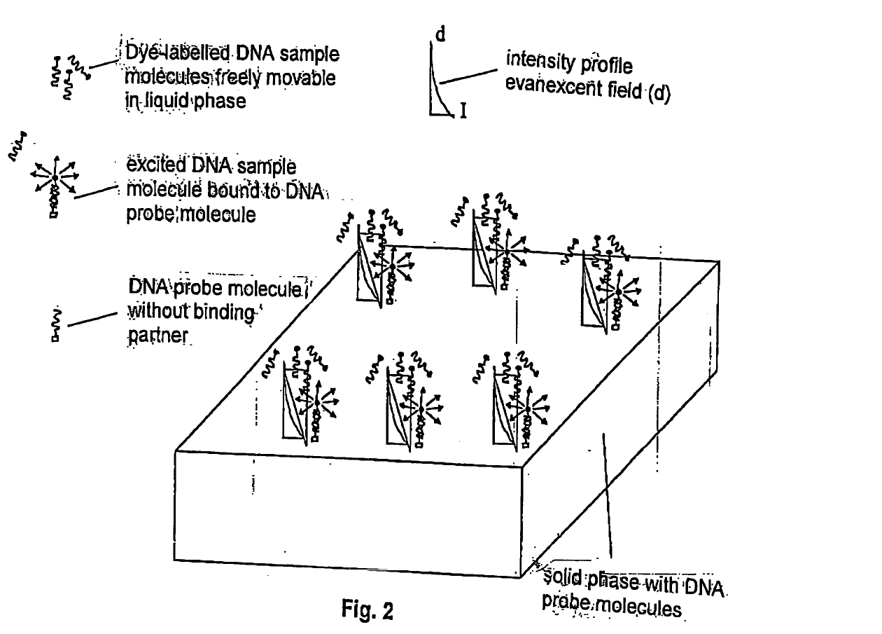

FIG. 2 shows a representation of the separation of bound and liquid phases according to the ATR principle;

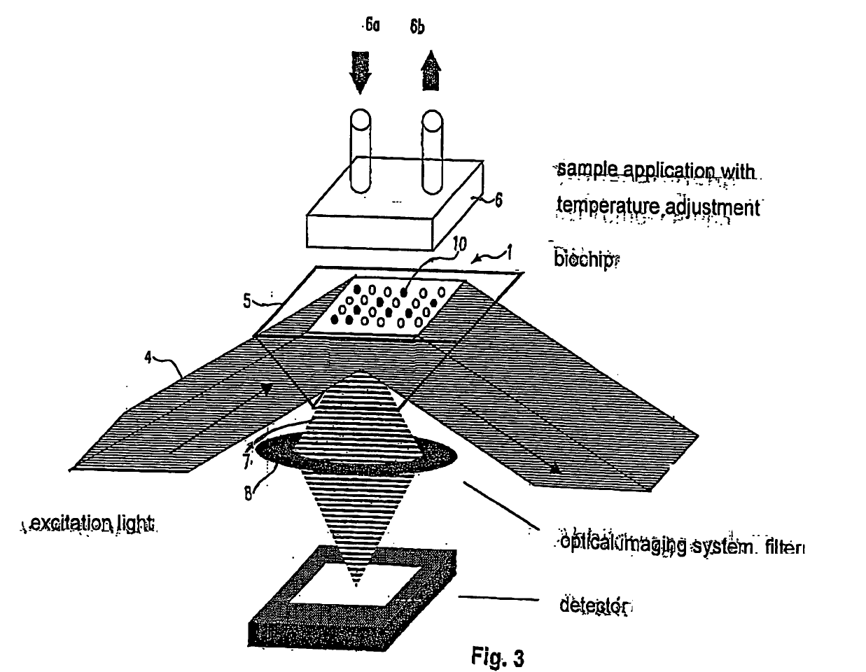

FIG. 3 shows a schematic representation of the device for reading an ATR prism by means of single reflection;

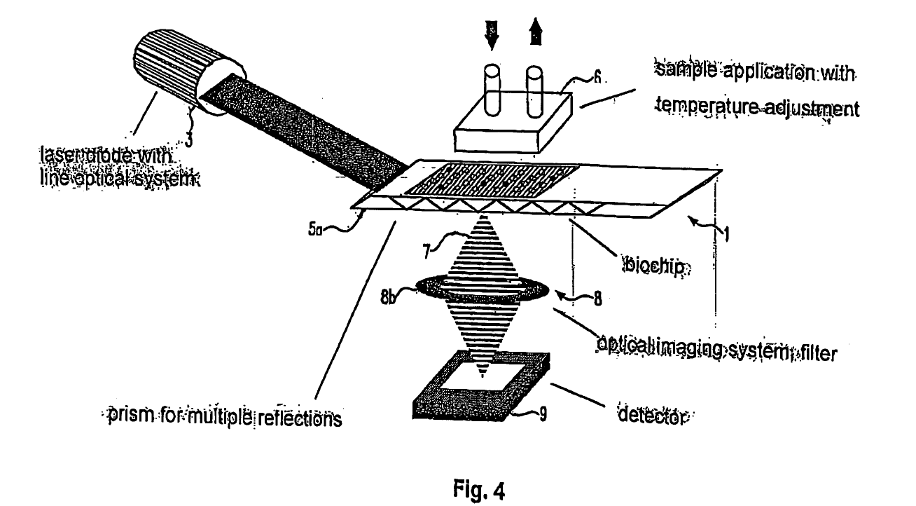

FIG. 4 shows a schematic representation for reading an ATR prism by means of multiple reflection;

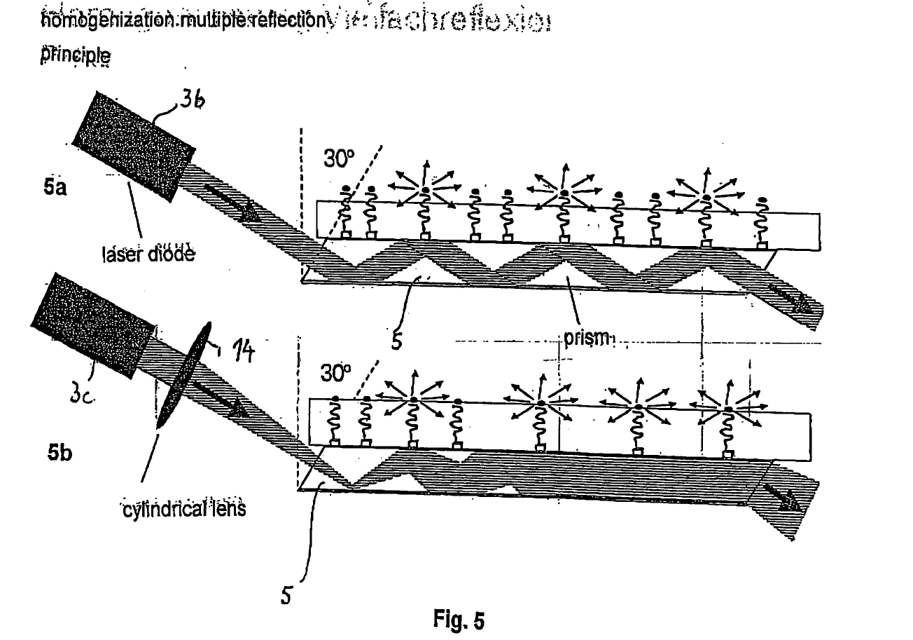

FIG. 5 shows a schematic representation of the device making use of the principle of a “homogenized” multiple reflection (area illumination);

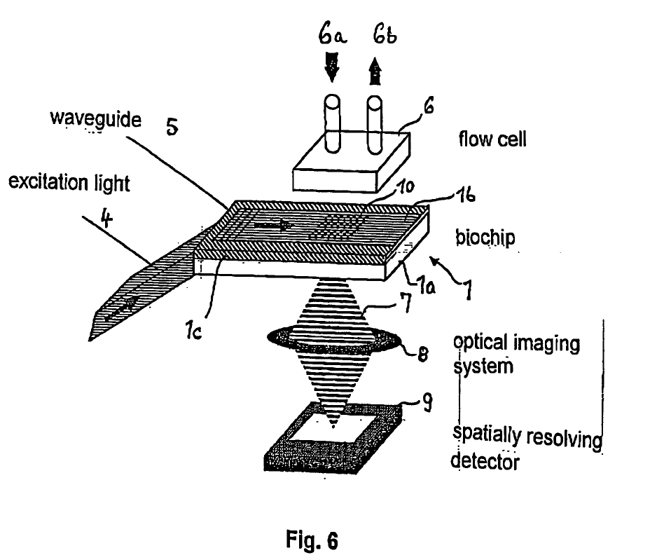

FIG. 6 shows a schematic representation of the device making use of a planar optical waveguide;

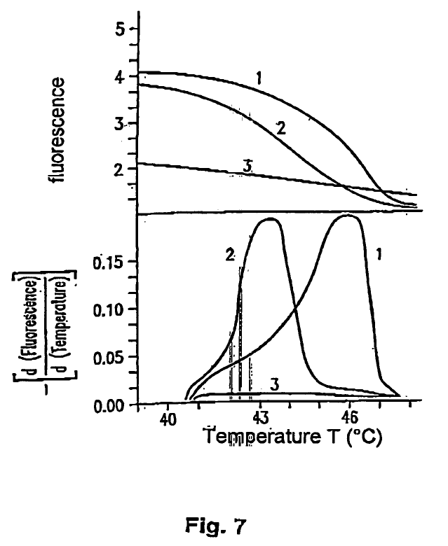

FIG. 7 shows a graph representing the fluorescence distribution as well as its derivation over time; and

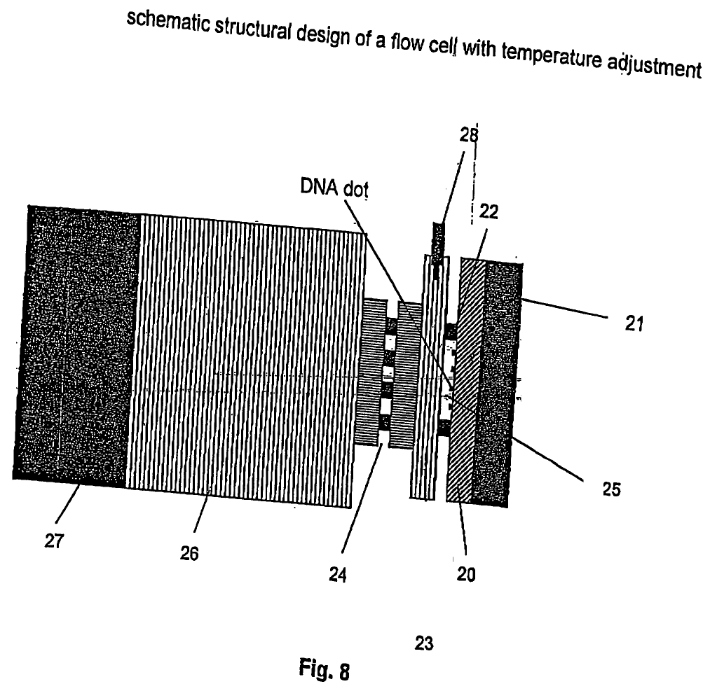

FIG. 8 shows a schematic structural design of a flow cell with temperature adjustment.

The design and the reading of a biochip is selected as first embodiment of the method and of the device constituting the subject matter of the present patent application, said biochip being used for the analysis of DNA sequences which are contained in a sample and which are contacted with a surface of a biochip so as to analyze the nucleic acid.

It goes without saying that this is only one embodiment and that also other molecular biological, biological and/or chemical substances, such as genes and antibodies, can be detected.

FIG. 1a shows a schematic representation of such a biochip 1 which forms a small platelet on the surface of which a plurality of nucleic acids 11 is immobilized at individual measuring points 10. At each individual measuring point 10, an oligonucleotide with a defined base sequence is present. This is shown in FIG. 1b. In FIG. 1c the nucleic acids of the test sample which are to be analyzed are designated by reference numeral 12 and, by means of an arrow, it is indicated that these nucleic acids are contacted with the complementary nucleic acids 11 located at the measuring point 10. Since each single strand of a nucleic acid molecule 11 binds to its complementary strand 12 (hybridization) (cf. FIG. 1b), information on the DNA sequences 12 existing in the sample will be obtained when the individual measuring points have been examined with respect to the binding of sample molecules 12. The hybridized DNA single strands are designated by reference numeral 13 in FIG. 1c.

The following embodiments use either the attenuated total reflection (ATR) or the total internal reflection fluorescence (TIRF). Due to the total reflection of a light beam on the Interface between two media having different optical densities, an electromagnetic field is produced in the optically lighter medium. The optically denser medium is here a solid phase and the optically lighter medium is a liquid phase. This so-called evanescent field penetrates only a few hundred nm from the interface into the liquid ambient medium. Hence, the dyes detected are almost exclusively the fluorescent dyes bound to the surface. The dyes dissolved in the ambient medium contribute to the measuring signal only to a minor extent, as shown in FIG. 2. This permits the measurement of the temporal progress of reactions.

In FIG. 2 It is clearly shown that the intensity profiles of the evanescent field drop steeply.

A heatable flow-through cell brings the liquid phase into contact with the solid phase and is adapted to be used for bringing the reaction partners to a specified temperature range. A flow cell 6 is coupled to a fluidic system for handling the liquid phase. Due to the permanent contact between the probe, i.e. the nucleic acid bound to the solid phase, and the liquid phase, the biochip is capable of being regenerated. The component used as a measuring chip is a transparent prism 5.

In the case of the embodiment according to FIG. 3, the edge of the prism 5 is illuminated in large area. A single total reflection at the base of the prism suffices to obtain a sufficiently large measuring area.

FIG. 3 additionally shows, in a schematic representation, a device for reading the biochip 1, said biochip having a configuration of the type which has already been described hereinbefore. The biochip 1 again comprises a transparent substrate and, preferably, a coating which has a high refractive index and which is applied to the substrate, said coating being used as a planar optical waveguide. The optical waveguide carries a field of measuring points 10, and excitation light 4 is coupled via the prism 5 into said optical waveguide and guided therein. The measuring probe is implemented in the way which has been explained making reference to FIG. 1b.

For analyzing DNA nucleic acids in a sample, the sample is here guided in the flow cell 6 and passed through said flow cell 6, as indicated by the flow arrows 6a and 6b The flow cell 6 is sealingly attached to the optical waveguide and encompasses the measurement field with the measuring points 10 in a framelike and fluid-tight manner so that the sample can interact with all measuring points 10 for a possible hybridization. The fluorescent radiation 7 excited by the evanescent field of the excitation light 4 is detected e.g. by means of an optical imaging system 8 in combination with a filter which is here not shown and a spatially-resolving detector 9, such as a CCD camera or a photomultiplier.

In this way, a detection of the hybridization and a parallel reading of all the measuring points 10 of the biochip 1 can be effected simultaneously. At the same time, a selective excitation of the bound fluorochromes takes place in the measuring points 10. It follows that, on the basis of this measurement principle without particular sample preparation, a very fast evaluation and detection of the biochip 1 is possible with great accuracy as far as the spatial resolution and also as far as the presence of hybridized nucleic acids is concerned.

In the case of the set-up according to FIG. 4, an edge of a much thinner prism 5a having a thickness of approx. 1 mm is illuminated by a line optical system. A plurality of prisms 5a are here arranged side by side. Due to multiple reflections at the upper and lower surfaces of said prisms 5a, a large-area illumination of the measuring field is produced. The emitted fluorescent radiation is detected by a spatially resolving detector 9. In this case, a laser diode 3 is used as a light source.

On-line measurement of the hybridization by means of an ATR analyzer provided with a heatable fluidic system is carried out as described hereinbelow.

To begin with, the determination of the melting point of a DNA will be described. In so doing, probes can be hybridized with synthetic samples, i.e. oligonucleotides, or with natural samples, i.e. cDNAs. Recording of the signal intensity at different temperatures permits a determination of the melting point Tm of the DNA. This is the temperature at which 50% of the maximum signal strength are reached. This test can be carried out with a large number of probes.

The determination of the formation constant will be described next. On the basis of a known concentration of molecules of the substance to be analyzed, the rate constant of a reaction can be measured in accordance with the law of mass action. This can be done on the chip with a large number of analysis points.

When the melting point and the formation constant are known, the concentration of one or of a plurality of analytes in a complex sample can be determined by recording the hybridization curve.

FIG. 5 shows in FIG. 5a and 5b the respective conditions under which light is radiated into a prism 5 used as a reaction carrier, the laser beam, which is radiated into the prism through a laser diode 3a, being represented as a laser beam with multiple reflections in the ATR prism 5 (FIG. 5a), whereas FIG. 5b shows the possibility of actually obtaining a large-area illumination of the surface of the prism 5 in that the light is radiated into the prism 5 by the light source 3c and through a cylindrical lens 14.

As can be seen, an actually substantially full-area illumination of the prism surface is achieved in this way, whereas, when a collimated laser beam is used for illuminating the edge of the prism 5, this has the effect that the upper and the lower surfaces of the prism 5 are illuminated selectively only at specific points and that measuring points which are located in the non-illuminated areas cannot be evaluated. If the light beam is, however, focused onto the edge of the prism 5 by means of a cylindrical lens 14, this will have the effect that the light beam spreads divergently in the prism 5. After a certain distance, the beam has been expanded to such an extent that a virtually homogeneous illumination of the biochip surface is given.

FIG. 6 shows in a schematic representation a device for reading a biochip 1, said biochip having a configuration of the type shown in FIG. 2. The biochip 1 again comprises the transparent substrate 1a and the coating which has a high refractive index and which is applied to the substrate, said coating being used as a planar optical waveguide 1b. The edges 1c of the biochip 1 remain outside of the waveguide structure. The optical waveguide carries a field of measuring points 10, and the excitation light 4 is coupled via a coupling grating 5 into said optical waveguide 1b and guided therein. The measuring points 10 are implemented in the way which has been explained making reference to FIG. 1b and FIG. 2. For analyzing DNA nucleic acids in a sample, the sample is here guided e.g. in a flow cell 6 and passed through said flow cell 6, as indicated by the flow arrows 6a and 6b. The flow cell 6 is sealingly attached to the optical waveguide 1b and encompasses the measurement field with the measuring points 10 in a framelike and fluid-tight manner so that the sample can Interact with all m assuring points 10 for a possible hybridization. The fluorescent radiation 7 excited by the evanescent field of the excitation light is detected e.g. by means of an optical imaging system 8 in combination with a filter (which is here not shown) and a spatially-resolving detector 9, such as a CCD camera or a photomultiplier. The detection can, however, also be carried out by emitting the fluorescent light upwards above the waveguide 1b.

In this way, an “in-situ detection” of the hybridization and a parallel reading of all the measuring points 10 of the biochip 1 can be effected simultaneously. At the same time, a selective excitation of the bound fluorochromes takes place in the measuring points. It follows that, on the basis of this measurement principle, a very fast evaluation and detection of the biochip 1 is possible with great accuracy as far as the spatial resolution and also as far as the presence of hybridized nucleic acids is concerned.

As is generally known, an analysis set-up according to FIG. 6 necessitates an additional outlay for coupling the excitation light 4 into the waveguide 1b (here via a grating 5). On the one hand, this necessitates additional preparation steps for the production of the biochip 1, and, on the other hand, adjustment devices are required in the optical set-up between the excitation light source (laser source) and the biochip. The resultant increase in the costs for the biochip, which is problematic since the biochip is a consumable material, can be avoided by using a measurement and analysis set-up according to the schematic representation according to FIG. 4 in the case of which excitation of the fluorescence is effected on the upper side of the optical waveguide 1b, e.g. from the back of the biochip 1 and, consequently, from the opposite side when seen in relation to the flow cell 6, whereas detection of the fluorescent light emitted by the bound fluorochromes is provided by coupling said fluorescent light into the planar waveguide 1b.

As an alternative solution, the excitation light emitted by a laser beam source is guided, preferably via a deflection unit 14, onto a reflecting mirror 15 and from said reflecting mirror into the waveguide 1b in the area of the measurement field of the measuring points 10 where e.g. the flow cell 6 is located. In the course of this action, a biaxial relative movement between the excitation light beam and the biochip is carried out with a scanning means. Also in this case, a separation between bound and dissolved fluorochromes is achieved by the planar waveguide 1b, since only the fluorescent light 7 emitted in the area of the evanescent field of the excitation light 4 is actually coupled Into the planar waveguide 1b and, subsequently, detected. The dissolved fluorochromes do not produce any background intensity, since this light is not routed to the detection means, the light routed to the detection means being only the fluorescent light of the bound fluorochromes which is guided in the planar waveguide 1b. The flow arrows 6a, 6b again indicate the sample flow through the flow cell 6, whereas the optical imaging system 8 with a filter is shown after the planar optical waveguide 1b, a photomultiplier being here used as a spatially-resolving detector.

Since line-by-line reading has to be effected in the case of this embodiment, the biochip 1 is moved accordingly in the direction of the arrow, as indicated.

If necessary, a detection means comprising an optical evaluation system, a filter and a photomultiplier can also be provided on the other side of the waveguide so as to detect the fluorescent light emerging from the optical waveguide 1b on the other side thereof, or the detector can be coupled directly to the edge of the optical waveguide 1b.

Also a glass plate can directly be used as an optical waveguide, said glass plate itself defining the reaction carrier and also the planar optical waveguide. In this case, a substrate need not be provided with a separate coating defining the optical waveguide.

The solution according to the present invention permits real-time measurement and evaluation of biochips or of other reaction carriers on whose upper surface, which is coated with a planar waveguide, analyses of substances in samples are effected by reaction.

Making reference to FIG. 7, a further embodiment will now be described.

By varying the temperature, the melting points of immobilized oligonucleotides can be determined, since dissociation or binding of the sample can be observed in response to an increase or decrease in temperature and since the melting point can be determined mathematically from the melting curve that can be produced on the basis of this observation. Melting point determination can be carried out for many oligonucleotides simultaneously in an incubation parallelized on the chip.

For determining the melting curve, oligonucleotides have, for example, been used whose sequence corresponds to a part of the haemochromatosis gene. Oligonucleotides of different lengths as well as different base substitutions were tested at various points. The synthetically produced oligonucleotides were provided at the 5′ end with a respective spacer consisting of 10 thymine bases and a C6 amino linker. Via the amino group on the linker, the oligonucleotides were covalently linked to silanized glass slides. For the purpose of detection, a hybridization was carried out either with a complementary, fluorescence-labelled (Cy5) oligonucleotide or a PCR product from haemochromatosis patients and the dissociation kinetics was measured in the ATR reader with an increase in temperature E.g. for oligonucleotides having a length of 17 bases which had been immobilized in a concentration of 2 μM on a glass chip and which contained at a central position either the base G (5′ ATATACGTGCCAGGTGG 3′; SEQ ID NO:1) corresponding to the wild type, which is represented by curve 1 of FIG. 7, or the base A (5′ ATATACGTACCAGGTGG 3′; SEQ ID NO:2) corresponding to the mutant, which is represented by curve 2 of FIG. 7, this measurement resulted in the following melting points in the case of a hybridization with a complementary equimolar oligonucleotide with a length of 31 bases at room temperature and a subsequent increase in temperature: the complementary oligonucleotide dissociated earlier (Tm 43° C.) from the oligonucleotides containing a missense base than from the oligonucleotide corresponding to the wild type (Tm 46° C.).

The fluorescence decreases due to the detachment of the fluorescence-labelling complementary oligonucleotide from the oligonucleotide probe when the temperature increases.

This is shown by curve 1 in the upper graph of FIG. 7 for the immobilized wild-type oligonucleotide, whereas curve 2 represents the immobilized oligonucleotide containing the missense base and corresponding to the mutant.

Curve 3 serves for the purpose of a check measurement and shows the hybridization without an oligonucleotide.

The lower graph of FIG. 7 shows the derivation of fluorescence over time. For the sake of clarity, the derivation has been plotted after a sign inversion.

In the following, an embodiment for determining the temperature dependence of the equilibrium constant of protein/protein and protein/ligand complexes is described.

The prism was silanized on both sides with an amino-silane group in accordance with known processes The proteins and ligands to be linked were activated making use of the carbo-diimide NHS process, which leads to activated carboxyl groups of the proteins and ligands.

The activated proteins and ligands were applied to the prism in a the form of an array by means of a pin printer. After said application, the prism was incubated in a moist chamber at 37° C. for two hours. The prism was then incubated in a borate buffer pH 9.5 at room temperature for 30 minutes so as to effect a hydrolysis of the residual active ester groups and, subsequently, in 1% BSA (w/v) in 100 mM PBS pH 7.4 at room temperature for one hour so as to block the prism surface against non-specific binding.

The analyte (proteins and ligands) were fluorescence labelled with the Cy5 labelling kit (Pharmacia) according to the manufacturer's instructions.

Subsequently, the prism was installed in the ATR detector element and the flow cell was rinsed with PBS and then filled with 1 mM fluorescence-labelled analyte. When the equilibrium state had been reached, a 30 s record was made. The temperature of the flow cell was increased stepwise by X° C. per minute. 30 s records were made after respective X-min intervals. When the desired temperature had been reached, the individual measuring points in the array were quantified making use of the SignalseDemo Software (GeneScan). For determining the temperature dependence of the equilibrium constant, a suitable regression function was incorporated into the measurement data with the aid of the program Grafit (Erithacus Software).

FIG. 8 shows how the flow cell is brought to a specified temperature range.

For recording DNA melting curves, it is necessary to bring the probe-sample hybrids in the flow cell to a homogeneous specified temperature range. The temperature-adjustment unit must be able to cover a large temperature range so that the melting point of very short and also of very long nucleotides can be measured. The temperature range between 0° C. and 100° C. can easily be realized by peltier heating/cooling. The use of a peltier element 24 also permits a very compact structural design. FIG. 8 shows the schematic structural design of the flow cell which is adapted to be brought to a specified temperature range. The biochip 20 is pressed onto the flow cell by means of a chip holder 21. A depression in the flow cell defines the reaction volume 25 which is sealed by an O-ring sealing means 22. The back 23 of the flow cell is contacted with a peltier element 24 so as to bring it to a specified temperature range. Heat exchange with the environment is realized by a copper block 26 with a blower 27. A resistance thermometer 28, which is installed in the flow cell; is used for measuring the temperature and forms together with a PID controller and the peltier element 24 a control circuit.

1. A method of determining temperature-dependent parameters, such as the association/dissociation parameters and/or the equilibrium constant of complexes that comprise at least two components, wherein the first components (12), which are in a liquid phase, are contacted with measuring points (10) located on an optically excitable reaction carrier and formed by second components (11) linked to the solid reaction carrier and specifically binding to said first components (12), under formation of complexes (13), wherein the excitation of fluorescent dyes which are bound to the first components (12) and/or the second components (11) and which are located close to the surface is effected by transmitted excitation light (4) so that fluorescent light (7) will be emitted, and the detection of the emitted fluorescent light takes place in a variable temperature field, and wherein the formation or the dissociation of the complexes comprising first components (12) and second components (11) is observed as a function of temperature.

2. A method according to claim 1, characterized in that the reaction carrier (1) is a biochip.

3. A method according to one of the preceding claims, characterized in that the first and/or second components are oligopeptides or polypeptides.

4. A method according to one of the preceding claims, characterized in that the first and/or second components are nucleic acid single strands.

5. A method according to one of the preceding claims, characterized in that the excitation light (4) is coupled into a preferably planar optical waveguide with the aid of optical means, such as one or a plurality of prisms (5; 5a).

6. A method according to one of the preceding claims, characterized in that, by total reflection (ATR) or total internal reflection fluorescence (TIRF) of the light beams at an interface between two media having different optical thicknesses, the excitation light produces an electromagnetic field in the optically lighter medium, the optically denser medium being a solid phase and the optically lighter medium a liquid phase, for measuring the temporal progress of the reaction.

7. A method according to claim 5 or 6, characterized in that prisms (5a) are provided, which, due to multiple reflections at the upper and lower surfaces of said prisms (5a), produce by means of a line optical system a large-area illumination of a measuring field.

8. A method according to at least one of the preceding claims 1 to 7, characterized in that, with the aid of excitation light (4) coupled into the planar optical waveguide, all measuring points (10) are excited simultaneously.

9. A method according to claim 6, characterized in that the fluorescent light (7) of the second components (11), which specifically bind to said first components (12), is supplied, preferably by means of an optical imaging system Including a filter (8a), to a spatially-resolving detector (9) above or below the biochip (1) or on the side, so as to read the biochip (1).

10. A method according to one of the preceding claims, characterized in that the liquid phase is degassed.

11. A method according to at least one of the claims 1 to 10, characterized in that the fluorescent light is produced by the evanescent field by means of excitation light, especially laser light, coupled into a planar optical waveguide.

12. A method according to at least one of the preceding claims 1 to 10, characterized In that the fluorescent light is produced by excitation light, especially laser light, from the environment of an optical element carrying the measuring points.

13. A device for determining temperature-dependent parameters, such as the association/dissociation parameters and/or the equilibrium constant of complexes that comprise at least two components, comprising a reaction carrier (1) whose optically excitable surface is provided with second components (11) specifically binding to the first components (12) and forming measuring points (10) on said reaction carrier, a device (6) for contacting the first components (12), which are in the liquid phase, and the second components (11) which are linked to the reaction carrier and which specifically bind to said first components, a means for bringing the measuring points to a specified temperature range, a light source (3) for coupling in excitation light (4) so as to excite the mission of fluorescent light (7) in dependence upon the binding of said first components (12) to said second components (11) of the reaction carrier (1), and a detector (9) for detecting the emitted fluorescent light (7) so as to determine the binding of said first components (12) to said second components (11) as a function of temperature.

14. A device according to claim 17, characterized in that the reaction carrier (1) is a biochip.

15. A device according to claim 12 or 13, characterized by optical means for coupling the excitation light (4) into an, especially planar optical waveguide.

16. A device according to claim 15, characterized in that the optical means are one or a plurality of prisms (5; 5a).

17. A device according to claim 16, characterized in that the prism or prisms (5; 5a) are implemented such that a single or multiple reflection of the light will take place.

18. A device according to claim 13 or 17, characterized in that the solid phase consists of glass or of a transparent plastic material.

19. A device according to at least one of the preceding claims 13 to 18, characterized by a degassing unit integrated in said device and used for degassing the liquid phase.

20. A device according to at least one of the preceding claims 13 to 17, characterized in that the device for contacting said first and second components with one another is heatable/coolable, especially heatable.

21. A device according to at least one of the preceding claims 13 to 18, characterized in that the device (6) for contacting said first components (12) with said second components (11) is a flow cell, a cuvette or a sample container disposed on the surface of the planar optical waveguide (1b) of the reaction carrier (1), in sealing connection therewith, in the area of the measuring points.

22. A device according to at least one of the preceding claims 13 to 21, characterized in that the reaction carrier is a biochip (1) with a planar optical waveguide on the upper surface thereof, which carries the measuring points (10).

23. A device according to at least one of the preceding claims 13 to 22, characterized in that the reaction carrier (1) is a glass plate, said glass plate itself forming the planar optical waveguide.

24. A device according to at least one of the preceding claims 13 to 23, characterized in that the excitation light (4) falls onto the reaction carrier from one side of said reaction carrier, and that the fluorescent light (7) emitted in the area of the evanescent field of the excitation light (4) by the fluorochromes bound to the surface of the planar optical waveguide is coupled Into the planar optical waveguide and guided therein, said fluorescent light (7) being adapted to be detected by the detection means (8; 9) arranged on at least one end face of the planar optical waveguide (1).

25. A device according to claim 25, characterized in that the detection means comprises an optical Imaging system (8) with a filter (8b) as well as a detector (9).

26. A device according to claim 25, characterized in that the detector (9) is a photomultiplier or a CCD camera.

27. A device according to at least one of the preceding claims 13 to 26, characterized in that a scanning means is provided for reading the reaction carrier (1) and that the reaction carrier (1) and/or the excitation light from the surroundings of the reaction carrier (1) is/are movable relative to said scanning means in at least one plane.

28. A device according to at least one of the claims 13 to 27, characterized in that the device for contacting said first and second components is heatable.

29. A device according to at least one of the preceding claims 13 to 28, characterized In that the reaction carrier carries an optical waveguide, especially a planar optical waveguide, on the surface of which the measuring points are provided.

30. A device according to one of the claims 13 to 29, characterized in that a biochip (20) is pressed onto a flow cell, and that a reaction volume (25), which is defined between said flow cell and said biochip, is sealed by an O ring.

31. A device according to claim 30, characterized in that a temperature adjustment means for said reaction volume is defined by a peltier element (24).

32. A device according to claim 31, characterized in that the peltier element is in contact with the back of the flow cell.

33. A device according to o of the claims 30 to 32, characterized in that a thermally conductive metal body, especially a copper block, is connected to said peltier element, the heat exchange of said conductive metal body with the environment being Influenced preferably by a subsequent blower element (27).

34. A device according to one of the claims 31 to 33, characterized in that the flow cell Includes a temperature sensor. especially a resistance thermometer, which, in combination with a controller, especially a PID controller, and the peltier element forms a control circuit.


SEQUENCE LISTING (2 sequences)

SEQ ID NO: 1 - DNA, 17 bp, Artificial
  Synthetic
atatacgtgccaggtgg

SEQ ID NO: 2 - DNA, 17 bp, Artificial
  Synthetic
atatacgtaccaggtgg